(19) United States
(12) Patent Application Publication
(10) Pub. No.: US 2026/0337580 A1
(43) Pub. Date: Oct. 29, 2026

(54) PROCESSES FOR CELL EXPANSION

(71) Applicant: SOCIÉTÉ DES PRODUITS NESTLÉ S.A., Vevey (CH)

(72) Inventors: Andy Wiranata WIJAYA, Ecublens (CH); Maria MARQUES DE LIMA, Prilly (CH); Marine KRAUS, Vuarrens (CH); Omid MASHINCHIAN, Lausanne (CH)

(21) Appl. No.: 19/123,148

(22) PCT Filed: Oct. 26, 2023
(86) PCT No.: PCT/EP2023/080004

§ 371 Date: Apr. 22, 2025

(30) Foreign Application Priority Data
Oct. 26, 2022 (EP) 22203766.5
Oct. 26, 2022 (EP) 22203772.3

(51) Int. Cl. C12N 5/074; A61K 35/545; C12M 1/06; C12N 5/00

(52) U.S. Cl. CPC C12N 5/0696; A61K 35/545; C12M 27/02; C12N 5/0062; C12N 2513/00; C12N 2527/00

(57) ABSTRACT

A process for the expansion of mammalian cells, the process comprising dynamically stirring cells in a bioreactor to produce an expanded cell culture, as well as products produced via said processes.

### FIELD OF THE INVENTION

The present invention concerns a process for the expansion of mammalian cells, the process comprising dynamically stirring cells in a bioreactor to produce an expanded cell culture, as well as products produced via said process and their uses.

### BACKGROUND OF THE INVENTION

Commercialization of cell-based technologies is a growing area of interest for multiple scientific and industrial fields. Stem cells in particular are an appealing cell type for biomedical and therapeutic applications. Researchers have made progress in providing processes for the large-scale manufacture of cells for use in these different fields, whilst keeping costs to a minimum. However, this is still a major commercial barrier.

Current technologies for large-scale manufacture of cells include the use of bioreactors. Bioreactors support the biological growth environment for cells, thus enabling cell expansion on a large-scale. In particular, stirred tank bioreactors are commonly used, as these bioreactors provide a 3-dimensional (3D), controlled environment compatible with different medium-changing protocols, which ultimately leads to improved cell growth.

However, cell expansion processes including those using stirred tank bioreactors still have various disadvantages. Recent studies have demonstrated that cell aggregates formed during expansion have large size variability. Increased cell aggregate size is associated with negative physical and physiological properties, including inefficient nutrient transport, reduced cell quality and viability.

The key to overcoming these problems is to control the size of the cell aggregates in the culture.

Accordingly, it is an object of the present invention to provide an improved process for cell expansion, where large-scale production is achieved with improved cell quality and viability.

### SUMMARY OF THE INVENTION

The present invention solves the above-mentioned technical problem.

Provided herein is a process for expanding mammalian cells in a stirred tank bioreactor, the bioreactor comprising cells and a cell culture medium, the process comprising dynamically stirring the cells in the bioreactor to produce an expanded cell culture.

In one embodiment, the dynamic stirring comprises stirring the cells at a stirring speed that is altered during production of the expanded cell culture.

In one embodiment, the stirring speed is increased from a first stirring speed to a second stirring speed.

In one embodiment, the stirring speeds are in the range of 150 to 800 RPM.

In one embodiment, the first stirring speed is increased to a second stirring speed at a rate of 6.25 to 20 RPM per hour.

In one embodiment, the first stirring speed is 150 RPM to 400 RPM, and optionally the second stirring speed is 450 RPM to 500 RPM.

In one embodiment, the first stirring speed is 250 RPM, and optionally the second stirring speed is 400 RPM.

In one embodiment, the stirring powers are in the range of 3 to 600 W/m³.

In one embodiment, the first stirring power is increased to a second stirring power at a rate of 2.35 to 23.53 W/m³ per hour.

In one embodiment, the first stirring speed is 3 W/m³ to 75 W/m³, and optionally the second stirring speed is 105 W/m³ to 150 W/m³.

In one embodiment, the first stirring power is about 18 W/m³ including 18.19 W/m³ and optionally the second stirring power is about 75 W/m³ including 74.52 W/m³.

In one embodiment, the dynamic stirring of the expansion process has a duration of 3 to 30 days, optionally 5 days.

In one embodiment, the stirring speed is altered from day 2 to day 3, where the expansion process is initiated at day 0.

In one embodiment, the cells for expansion are human cells, and/or wherein the cells for expansion are stem cells.

In one embodiment, the cells for expansion are selected from any one of pluripotent stem cells (PSCs), muscle stem cells/satellite cells (MuSC/SC), adipose derived stem cells (ADSC), adipocytes, epithelial cells, mesenchymal stem/stromal cells (MSCs), fibro-adipogenic progenitors (FAPS), induced-pluripotent stem cells (iPSCs), breast milk stem cells (BMSCs), embryonic-like stem cells (ELC-Cs), chemically induced pluripotent stem cells (CiPSCs) and chemically induced totipotent stem cells (CiTotiSCs), optionally wherein the cells for expansion are iPSCs.

Also provided herein is a process for expanding mammalian cells in a stirred tank bioreactor comprising:
i) inoculating the bioreactor with a single-cell sample,
ii) dynamically stirring the cells in the bioreactor, and
iii) isolating the expanded cell culture.

In some embodiments, the mammalian cells are mammalian pluripotent stem cells (PSCs).

In one embodiment, the process comprises:
i) inoculating the bioreactor with a single-cell sample,
ii) dynamically stirring the cells in the bioreactor, wherein:
   a) the cells are stirred at a first stirring speed, optionally of 250 RPM from day 0 to day 2,
   b) the first stirring speed is increased to a second stirring speed, optionally at a rate of 6.25 RPM per hour from day 2 to day 3, and
   c) the cells are stirred at a second stirring speed, optionally of 400 RPM from day 3 to day 5,
iii) isolating the expanded cell culture after 5 days.

In one embodiment, the process comprises:
i) inoculating the bioreactor with a single-cell sample of PSCs, wherein the single-cell sample is a sample of iPSCs,
ii) dynamically stirring the cells in the bioreactor, wherein:
   a) the cells are stirred at a first stirring speed, optionally of 250 RPM from day 0 to day 2,
   b) the first stirring speed is increased to a second stirring speed, optionally at a rate of 6.25 RPM per hour from day 2 to day 3, and
   c) the cells are stirred at a second stirring speed, optionally of 400 RPM from day 3 to day 5,
iii) isolating the expanded cell culture after 5 days.

In one embodiment, the process comprises:
i) inoculating the bioreactor with a single-cell sample of PSCs, wherein the single-cell sample is a sample of iPSCs,
ii) dynamically stirring the cells in the bioreactor, wherein:
   a) the cells are stirred at a first stirring speed, optionally of 150 RPM to 400 RPM from day 0 to day 2,
   b) the first stirring speed is increased to a second stirring speed, optionally at a rate of 6.25 RPM per hour from day 2 to day 3, and
   c) the cells are stirred at a second stirring speed, optionally of 400 RPM to 500 RPM from day 3 to day 5, wherein the first and second stirring speeds are different,
iii) isolating the expanded cell culture after 5 days.

In one embodiment, the process comprises:
i) inoculating the bioreactor with a single-cell sample,
ii) dynamically stirring the cells in the bioreactor, wherein:
   a) the cells are stirred at a first stirring power, optionally of about 18 W/m³ including 18.19 W/m³, from day 0 to day 2,
   b) the first stirring power is increased to a second stirring speed, optionally at a rate of 2.35 W/m³ per hour from day 2 to day 3, and
   c) the cells are stirred at a second stirring power, optionally of about 75 W/m³, including 74.52 W/m³, from day 3 to day 5,
iii) isolating the expanded cell culture after 5 days.

In one embodiment, the process comprises:
i) inoculating the bioreactor with a single-cell sample of PSCs, wherein the single-cell sample is a sample of iPSCs,
ii) dynamically stirring the cells in the bioreactor, wherein:
   a) the cells are stirred at a first stirring power, optionally of about 18 W/m³ including 18.19 W/m³ from day 0 to day 2,
   b) the first stirring speed is increased to a second stirring power, optionally at a rate of 2.35 W/m³ per hour from day 2 to day 3, and
   c) the cells are stirred at a second stirring power, optionally of about 75 W/m³ including 74.52 W/m³, from day 3 to day 5,
iii) isolating the expanded cell culture after 5 days.

In one embodiment, the process comprises:
i) inoculating the bioreactor with a single-cell sample of PSCs, wherein the single-cell sample is a sample of iPSCs,
ii) dynamically stirring the cells in the bioreactor, wherein:
   a) the cells are stirred at a first stirring power, optionally of 3 to 75 W/m³ from day 0 to day 2,
   b) the first stirring speed is increased to a second stirring power, optionally at a rate of 2.35 W/m³ per hour from day 2 to day 3, and
   d) the cells are stirred at a second stirring power, optionally of 75 to 150 W/m³ from day 3 to day 5, wherein the first and second stirring powers are different,
iii) isolating the expanded cell culture after 5 days.

In one embodiment, the process produces cell aggregates, wherein the process decreases cell aggregate size compared to a process which does not comprise dynamic stirring.

In one embodiment, the process increases the circularity of the cell aggregates compared to a process which does not comprise dynamic stirring.

In one embodiment, the process increases cell viability compared to a process which does not comprise dynamic stirring.

In one embodiment, the cells for expansion are pluripotent cell, and the process increases cell pluripotency compared to a process which does not comprise dynamic stirring.

In one embodiment, the expanded cell culture is suitable for use as a food product or as a medicine.

Finally, provided herein is an expanded cell culture produced according to any process of the invention described herein, optionally wherein the cells are PSC.

### Detailed Description of the Invention

### DEFINITIONS

Within the context of the present invention, the term “cell expansion” (or similar) refers to the process by which cells are cultivated to produce a larger number of cells of the same cell type. The term “cell expansion” may be used interchangeably with “cell cultivation”.

Within the context of the present invention, the term “stirring speed” refers to rotational speed. In some instances, stirring speed is described as the revolutions per minute (RPM). In some instances, stirring speed may be represented as stirring power in power per volume (P/V). Power may be measured in Watts (W). Volume may be measured in m³. Stirring speed may be converted to stirring power, the formula for which is described in Rotondi, M. et al. 2021 which is incorporated herein by reference (Rotondi, M. et al. Design and development of new ambr250® bioreactor vessel for improved cell and gene therapy application. Biotechnology Letter 43, 1103-1116, doi:10.1007/s10529-021-03076-3 (2021)).

Within the context of the present invention, the term “dynamically stirred” (or similar) refers to the process of altering a characteristic of stirring.

Within the context of the present invention, the term “statically stirred” (or similar) refers to the process of stirring at a single speed, wherein the speed is not altered.

Within the context of the present invention, the term “cell viability” refers to healthy, live cells. Viability may be calculated as the % number of cells in a sample that are healthy.

Within the context of the present invention, the term “pluripotency” refers to the ability of a cell to develop into the three primary germ cell layers of the early embryo and therefore into all cells of the adult body.

Within the context of the present invention, the term “seed culture” refers to a small sample of viable single-cells.

Within the context of the present invention, the term “culture medium” refers to any suitable medium that enables cell expansion of the cell type of interest. The culture medium can comprise basal media (e.g. DMEM and/or F12) and growth factors, for example, fibroblast growth factor, insulin, transferrin and/or transforming growth factor beta. For example, in the case of an induced pluripotent stem cell expansion process, the culture medium may comprise mTeSR™. mTeSR™ is serum-free and comprises basal medium and recombinant human basic fibroblast growth factor and recombinant human transforming growth factor β. An exemplary culture medium is described in Kuo etal. 2020, which is herein incorporated by reference (Kuo, H. H., Gao, X., DeKeyser, J. M., Fetterman, K. A., Pinheiro, E. A., Weddle, C. J. & Burridge, P. W. (2020). Negligible-cost and weekend-free chemically defined human iPSC culture. Stem Cell Reports, 14(2), 256-270).

Within the context of the present invention, the term “cell circularity” refers to the morphological roundness of a cell aggregate and may be quantified by any known methods in the art, such as by using an image processing algorithm, for example NIS Elements software. Cell circularity ranges from 0 to 1 (arbitrary units), where 1 is a perfect circle.

Within the context of the present invention, the term “cell aggregate” (or similar) refers to a cluster of adhered cells of a same cell type.

### Process According to the Present Invention

The present invention relates to processes for cell expansion, wherein cell quality and viability is improved compared to expansion processes described in the art, optionally wherein the cell is PSC.

During the expansion process, the expanding cells form cell aggregates. If these aggregates become too large, this can disrupt nutrient transport to the cells and cause build-up of metabolic waste products from the cells, which negatively impacts cell quality and viability.

In the present invention, the cells are dynamically stirred within a bioreactor during the expansion process. Preferably, the stirring speed is increased during the expansion process, such as from a first speed to a second speed.

It has been surprisingly demonstrated in the present invention that utilising a dynamic stirring process controls the aggregation size of the expanding cells, such that smaller cell aggregates are formed, with a more homogenous size distribution and improved circularity.

It has also been surprisingly demonstrated in the present invention that utilising a dynamic stirring process during cell expansion improves cell viability.

Further, it has been surprisingly demonstrated in the present invention that utilising a dynamic stirring process during cell expansion of pluripotent cells improves cell pluripotency.

Stirring speeds are associated with shear stress, and shear stress on cells during an expansion process is known to negatively impact cell properties such as quality and viability. It is therefore surprising that altering stirring speeds during a cell expansion process such as in the present invention does not negatively impact the cells, and further, actually improves cell quality and viability.

Thus, the invention provides a process for expanding mammalian cells in a stirred tank bioreactor, the bioreactor comprising cells and a cell culture medium, the process comprising dynamically stirring the cells in the bioreactor to produce an expanded cell culture.

In some embodiments, the process comprises:
i) inoculating a bioreactor with a single-cell sample,
ii) dynamically stirring the contents of the bioreactor, and
iii) isolating the expanded cell culture.

In some embodiments, the process comprises:
i) inoculating a bioreactor with a single-cell sample of PSCs,
ii) dynamically stirring the contents of the bioreactor, and
iii) isolating the expanded cell culture.

In some embodiments, the process comprises first culturing a cell of interest to produce a seed cell culture of single cells. Optionally, the cell is a PSC. In some embodiments, the culturing is conducted in 2-dimensional (2D) planar culture, such as a 2D T-flask. In some embodiments, the process comprises inoculating the bioreactor with the seed cell culture. In some embodiments, the bioreactor is inoculated with 50,000 to 5,000,000 cells per ml of the bioreactor vessel. In some embodiments, the bioreactor is inoculated with 50,000 to 5,000,000 cells per ml of the bioreactor vessel; 150,000 to 1,000,000 cells per ml of the bioreactor vessel; 200,000 to 500,000 cells per ml of the bioreactor vessel; or 250,000 to 500,000 cells per ml of the bioreactor vessel.

In some embodiments, the bioreactor is inoculated with 200,000 to 400,000 cells per ml of the bioreactor vessel.

The process comprises expanding cells in the bioreactor for a specific time period as described anywhere herein. In some embodiments, the process comprises isolating the expanded cells. The expanded cells are in the form of cell aggregates. In some embodiments, the cells have expanded by at least 10 fold. In some embodiments, the cells have expanded by at least 10 fold in 4 days. In some embodiments, the expanded cells are isolated by centrifugation. In some embodiments, the expanded cells are isolated by gravimetry. In some embodiments, the expanded cells are dissociated into single-cells for further expansion. In some embodiments, the expanded cells are dissociated into single-cells for storage in a biobank. In some embodiments, the expanded cells are exposed to differentiation media.

Certain features of the process of the invention will now be described in more detail.

### Dynamic Stirring

The process of the present invention requires dynamic stirring of cells in a bioreactor.

In one embodiment, the dynamic stirring comprises altering the stirring speed during the expansion process. In one embodiment, the stirring speed is altered more than once, such as two, three or four times during the expansion process. In one embodiment, the stirring speed is altered from a first stirring speed to a second stirring speed. In one embodiment, the stirring speed is gradually altered from a first stirring speed to a second stirring speed.

In one embodiment, the stirring speed is increased during the expansion process. In one embodiment, the stirring speed is increased more than once, such as two, three or four times during the expansion process. In one embodiment, the stirring speed is increased from a first stirring speed to a second stirring speed. In one embodiment, the stirring speed is gradually increased from a first stirring speed to a second stirring speed.

In some embodiments, the stirring speeds are 150 to 800 RPM. In some embodiments, the stirring speed is altered in the range of 150 to 800 RPM. In some embodiments, each stirring speed is selected from the range of 150 to 800 RPM.

In some embodiments, the stirring speeds are 200 to 800 RPM. In some embodiments, the stirring speed is altered in the range of 200 to 800 RPM. In some embodiments, each stirring speed is selected from the range of 200 to 800 RPM.

In some embodiments, the first stirring speed is 150 to 400 RPM. In some embodiments, the first stirring speed is 150 to 300 RPM. In some embodiments, the first stirring speed is 200 RPM to 400 RPM. In some embodiments, the first stirring speed is 200 to 300 RPM. In some embodiments, the first stirring speed is 225 to 275 RPM. In some embodiments, the first stirring speed is selected from 150 RPM, 200 RPM, 250 RPM, 300 RPM, 350 RPM and 400 RPM. In a preferred embodiment, the first stirring speed is 250 RPM.

In some embodiments, the second stirring speed is 300 to 800 RPM. In some embodiments, the second stirring speed is 300 to 600 RPM. In some embodiments, the second stirring speed is 350 to 450 RPM. In some embodiments, the second stirring speed is selected from 300 RPM, 350 RPM, 400 RPM, 450 RPM, 500 RPM, 550 RPM, 600 RPM, 650 RPM, 700 RPM, 750 RPM, and 800 RPM. Preferably, the second stirring speed is selected from 350 RPM, 400 RPM, and 450 RPM. In a preferred embodiment, the second stirring speed is 400 RPM to 500 RPM, including 400 RPM.

In some embodiments, the first stirring speed is 150 to 400 RPM which is increased during the expansion process to a second stirring speed of 300 to 800 RPM, wherein the first and second stirring speeds are different.

In some embodiments, the first stirring speed is 150 to 300 RPM which is increased during the expansion process to a second stirring speed of 300 to 800 RPM, wherein the first and second stirring speeds are different.

In some embodiments, the first stirring speed is 150 to 400 RPM which is increased during the expansion process to a second stirring speed of 300 to 800 RPM, wherein the first and second stirring speeds are different.

In some embodiments, the first stirring speed is 150 to 400 RPM which is increased during the expansion process to a second stirring speed of 300 to 600 RPM, wherein the first and second stirring speeds are different.

In some embodiments, the first stirring speed is 200 to 300 RPM which is increased during the expansion process to a second stirring speed of 300 to 500 RPM, wherein the first and second stirring speeds are different.

In some embodiments, the first stirring speed is less than 300 RPM and the second stirring speed is more than 300 RPM.

In some embodiments, the first stirring speed is selected from 150 RPM, 200 RPM, 250 RPM, 300 RPM and 400 RPM; and the second stirring speed is selected from 350 RPM, 400 RPM, 450 RPM, 500 RPM, 550 RPM and 600 RPM.

In some embodiments, the first stirring speed is 250 RPM and the second stirring speed is 400 RPM.

In some embodiments, the first stirring speed is increased to the second stirring speed at a rate of 6.25 to 20 RPM per hour. In a preferred embodiment, the first stirring speed is increased to the second stirring speed at a rate of 6.25 RPM per hour.

In some embodiments, the first stirring speed is 150 to 400 RPM, which is increased at a rate of 6.25 to 20 RPM per hour to a second stirring speed of 300 to 800 RPM.

In some embodiments, the first stirring speed is 150 to 300 RPM, which is increased at a rate of 6.25 to 20 RPM per hour to a second stirring speed of 300 to 800 RPM.

In some embodiments, the first stirring speed is 150 to 400 RPM, which is increased at a rate of 6.25 to 20 RPM per hour to a second stirring speed of 300 to 600 RPM.

In some embodiments, the first stirring speed is 200 to 300 RPM, which is increased at a rate of 6.25 to 20 RPM per hour to a second stirring speed of 300 to 500 RPM.

In a preferred embodiment, the first stirring speed is 250 RPM, which is increased at a rate of 6.25 RPM per hour to a second stirring speed of 400 RPM.

In some embodiments, the stirring powers are 3 to 600 W/m³. In some embodiments, the stirring power is altered in the range of 3 to 600 W/m³. In some embodiments, each stirring power is selected from the range of 3 to 600 W/m³.

In some embodiments, the stirring powers are 3.93 to 596.16 W/m³. In some embodiments, the stirring power is altered in the range of 3.93 to 596.16 W/m³. In some embodiments, each stirring power is selected from the range of 3.93 to 596.16 W/m³.

In some embodiments, the stirring powers are 9 to 600 W/m³. In some embodiments, the stirring power is altered in the range of 9 to 600 W/m³. In some embodiments, each stirring power is selected from the range of 9 to 600 W/m³.

In some embodiments, the stirring powers are 9.32 to 596.16 W/m³. In some embodiments, the stirring power is altered in the range of 9.32 to 596.16 W/m³. In some embodiments, each stirring power is selected from the range of 9.32 to 596.16 W/m³.

In some embodiments, the first stirring power is 3 to 75 W/m³. In some embodiments, the first stirring power is 3 to 32 W/m³. In some embodiments, the first stirring power is 9 to 75 W/m³. In some embodiments, the first stirring power is 9 to 32 W/m³. In some embodiments, the first stirring power is 13 to 24 W/m³. In some embodiments, the first stirring power is selected from 3 W/m³, 9 W/m³, 18 W/m³, 31 W/m³ and 74 W/m³. In a preferred embodiment, the first stirring power is about 18 W/m³ including 18.19 W/m³.

In some embodiments, the first stirring power is 3.93 to 74.52 W/m³. In some embodiments, the first stirring power is 3.93 to 31.44 W/m³. In some embodiments, the first stirring power is 9.32 to 74.52 W/m³. In some embodiments, the first stirring power is 9.32 to 31.44 W/m³. In some embodiments, the first stirring power is 13.26 to 24.22 W/m³. In some embodiments, the first stirring power is selected from 3.93 W/m³, 9.32 W/m³, 18.19 W/m³, 31.44 W/m³ and 74.52 W/m³.

In some embodiments, the second stirring power is 30 to 600 W/m³. In some embodiments, the second stirring power is 30 to 255 W/m³. In some embodiments, the second stirring power is 48 to 108 W/m³. In some embodiments, the second stirring power is selected from 31 W/m³, 50 W/m³, 74 W/m³, 106 W/m³, 145 W/m³, 193 W/m³, 251 W/m³, 319 W/m³, 399 W/m³, 491 W/m³, and 596 W/m³. Preferably, the second stirring power is selected from 49 W/m³, 74 W/m³, and 106 W/m³. In a preferred embodiment, the second stirring power is 74 to 145 W/m³ including 74 W/m³.

In some embodiments, the second stirring power is 31.44 to 596.16 W/m³. In some embodiments, the second stirring power is 31.44 to 251.51 W/m³. In some embodiments, the second stirring power is 49.92 to 106.1 W/m³. In some embodiments, the second stirring power is selected from 31.44 W/m³, 49.92 W/m³, 74.52 W/m³, 106.1 W/m³, 145.55 W/m³, 193.72 W/m³, 251.51 W/m³, 319.77 W/m³, 399.38 W/m³, 491.22 W/m³, and 596.16 W/m³. Preferably, the second stirring power is selected from 49.92 W/m³, 74.52 W/m³, and 106.1 W/m³. In a preferred embodiment, the second stirring power is 74.52 to 145.55 W/m³ including 74.52 W/m³. In some embodiments, the first stirring power is 3 to 75 W/m³ which is increased during the expansion process to a second stirring power of 30 to 600 W/m³, wherein the first and second stirring powers are different.

In some embodiments, the first stirring power is 3.93 to 74.52 W/m³ which is increased during the expansion process to a second stirring power of 31.44 to 596.16 W/m³, wherein the first and second stirring powers are different.

In some embodiments, the first stirring power is 18 to 75 W/m³ which is increased during the expansion process to a second stirring power of 30 to 600 W/m³, wherein the first and second stirring powers are different.

In some embodiments, the first stirring power is 18.19 to 74.52 W/m³ which is increased during the expansion process to a second stirring power of 31.44 to 596.16 W/m³, wherein the first and second stirring powers are different.

In some embodiments, the first stirring power is 3 to 32 W/m³ which is increased during the expansion process to a second stirring power of 32 to 600 W/m³, wherein the first and second stirring powers are different.

In some embodiments, the first stirring power is 3.93 to 31.44 W/m³ which is increased during the expansion process to a second stirring power of 31.44 to 596.16 W/m³, wherein the first and second stirring powers are different.

In some embodiments, the first stirring power is 9 to 32 W/m³ which is increased during the expansion process to a second stirring power of 32 to 150 W/m³, wherein the first and second stirring powers are different.

In some embodiments, the first stirring power is 9.32 to 31.44 W/m³ which is increased during the expansion process to a second stirring power of 31.44 to 145.55 W/m³, wherein the first and second stirring powers are different.

In some embodiments, the first stirring power is less than 30 W/m³ and the second stirring power is more than 30 W/m³.

In some embodiments, the first stirring power is less than 31.44 W/m³ and the second stirring power is more than 31.44 W/m³.

In some embodiments, the first stirring power is selected from about 3 W/m³, 9 W/m³, 18 W/m³, 31 W/m³ and 74 W/m³; and the second stirring power is selected from about 50 W/m³, 74 W/m³, 106 W/m³, 145 W/m³, 193 W/m³ and 251 W/m³.

In some embodiments, the first stirring power is selected from 3.93 W/m³, 9.32 W/m³, 18.19 W/m³, 31.44 W/m³ and 74.52 W/m³; and the second stirring power is selected from 49.92 W/m³, 74.52 W/m³, 106.1 W/m³, 145.55 W/m³, 193.72 W/m³ and 251.51 W/m³.

In some embodiments, the first stirring power is about 18 W/m³ including 18.19 W/m³ and the second stirring power is about 75 W/m³ including 74.52 W/m³.

In some embodiments, the first stirring power is increased to the second stirring power at a rate of 2.35 to 23.53 W/m³ per hour. In a preferred embodiment, the first stirring power is increased to the second stirring power at a rate of 2.35 W/m³ per hour.

In some embodiments, the first stirring power is 3 to 32 W/m³, which is increased at a rate of 2.35 to 23.53 W/m³ per hour to a second stirring power of 32 to 600 W/m³.

In some embodiments, the first stirring power is 3.93 to 31.44 W/m³, which is increased at a rate of 2.35 to 23.53 W/m³ per hour to a second stirring power of 31.44 to 596.16 W/m³.

In some embodiments, the first stirring power is 9 to 32 W/m³, which is increased at a rate of 2.35 to 23.53 W/m³ per hour to a second stirring power of 32 to 150 W/m³.

In some embodiments, the first stirring power is 9.32 to 31.44 W/m³, which is increased at a rate of 2.35 to 23.53 W/m³ per hour to a second stirring power of 31.44 to 145.55 W/m³.

In a preferred embodiment, the first stirring power is about 18 W/m³ including 18.19 W/m³, which is increased at a rate of 2.35 W/m³ per hour to a second stirring power of about 75 W/m³, including 74.52 W/m³.

The first and second stirring speeds/powers are different. The first and second stirring speeds can differ by at least 50 RPM, 100 RPM, 150 RPM or 200 RPM. The first and second stirring powers can differ by at least 45 W/m³, 50 W/m³, 60 W/m³, 70 W/m³ or 80 W/m³.

The dynamic stirring step of the expansion process is conducted over a certain numbers of days as described anywhere herein. The dynamic stirring step as described anywhere herein includes the duration whereby the cells are being stirred.

The dynamic stirring step is initiated on day 0.

In one embodiment, the duration of the dynamic stirring step is 3 to 30 days, such as 3 days, 4 days, 5 days, 7 days, 10 days, 15 days, 25 days or 30 days. Preferably, the duration is 5 days.

In one embodiment, the cells are stirred at the first stirring speed for at least 2 days.

In one embodiment, the cells are stirred at the first stirring speed for 2 days. In one embodiment, the cells are stirred at the first stirring speed from day 0 to day 2 (i.e., for 48 hours).

In one embodiment, the stirring speed is altered at least on day 2, such as on day 2, day 3, day 4 and/or day 5, preferably on day 3.

In one embodiment, the stirring speed is gradually altered for at least 1 day, such as for 1 day, 2 days or 3 days, preferably 1 day (i.e., for 24 hours). In this way, the stirring speed is gradually altered from a first speed to a second speed.

In one embodiment, the stirring speed is altered from day 2 to day 3. In one embodiment, the stirring speed is altered for 24 hours, from day 2 to day 3.

In one embodiment, the cells are stirred at the second stirring speed for at least 2 days.

In one embodiment, the cells are stirred at the second stirring speed for 2 days. In one embodiment, the cells are stirred at the second stirring speed from day 3 to day 5. Preferably, in one embodiment, the duration of the dynamic stirring is 5 days in total, and the stirring speed is increased from a first stirring speed to a second stirring speed for 24 hours, from day 2 to day 3.

In one embodiment, the expansion process is an iterative process, i.e., the process is repeated.

It will be understood that any features of the stirring speeds described anywhere herein may be combined with any features of the durations described anywhere herein.

For example, in one embodiment, the duration of the dynamic stirring is 5 days in total and the stirring speed is increased during the expansion process. In another embodiment, the duration of the dynamic stirring is 5 days in total and the stirring speed is increased from a first stirring speed to a second stirring speed. In another embodiment, the stirring speed is increased from a first stirring speed to a second stirring speed from day 2 to day 3. In another embodiment, the duration of the dynamic stirring is 5 days, wherein the stirring speed is increased from a first stirring speed of 250 RPM to a second stirring speed of 400 RPM from day 2 to day 3 at a rate of 6.25 RPM per hour. In another embodiment, the duration of the dynamic stirring is 5 days, wherein the stirring power is increased from a first stirring speed of about 18 W/m³, including 18.19 W/m³ to a second stirring speed of about 75 W/m³ including 74.52 W/m³ from day 2 to day 3 at a rate of 2.35 W/m³ per hour.

### Cell Types

The process of the present invention relates to expanding mammalian cells.

Cells for use in the present invention may include any mammalian cell type that is required to be expanded.

In one embodiment, the cells are human cells. In one embodiment, the cells are bovine cells. In one embodiment, the cells are stem cells. In one embodiment, the cells are human stem cells. In one embodiment, the cells are bovine stem cells. In one embodiment, the cells are adult human stem cells.

In one embodiment, the cells may be selected from any one of pluripotent stem cells (PSCs), muscle stem cells/satellite cells (MuSC/SC), adipose derived stem cells (ADSC), adipocytes, epithelial cells, mesenchymal stem/stromal cells (MSCs), fibro-adipogenic progenitors (FAPS), induced-pluripotent stem cells (iPSCs), breast milk stem cells (BMSCs), embryonic-like stem cells (ELC-Cs), chemically induced pluripotent stem cells (CiPSCs) and chemically induced totipotent stem cells (CiTotiSCs).

In a preferred embodiment, the cells are iPSCs. In another preferred embodiment, the cells are BMSCs. In a further preferred embodiment, the cells are epithelial cells.

It will be understood that the features of the cell type as described herein may be combined with any other features of the process of the invention, such as the dynamic stirring features as described anywhere herein.

### Cell Culture Vessel and Conditions

The process of the present invention relates to cell expansion in a cell culture vessel, specifically a stirred tank bioreactor.

To achieve stirring of the cell culture, the bioreactor may comprise one or more impellers. Preferably, the impeller is a single ‘elephant ear’ impeller. A single ‘elephant ear’ impeller having a 30 mm diameter and 45° pitched-blade angle is described in by Rotondi, M et al. 2021 (incorporated herein by reference) and may be used in any embodiments of the present invention described herein. Hence, the power number (Np) to convert the stirring speed to PPV is 2.07.

In one embodiment, the bioreactor comprises one or more bioreactors chambers. In some embodiments, the reactor chamber has an internal volume of 0.1-100,000 L. In some embodiments, the working volume is up to 50,000 L, including 100 mL to 50,000 L and up to 10,000 L, including 100 mL to 10,000 L. The working volume can also be 100 to 500 mL, 150 to 400 mL, 200 to 300 mL, or 200 to 250 mL.

In some embodiments, the fluid density is 800 to 1,200 kg/m³ of aqueous solution. Preferably, the fluid density is 1,000 kg/m³ of aqueous solution.

In some embodiments, the bioreactor is inoculated with the seed cell culture. In some embodiments, the bioreactor is inoculated with 50,000 to 5,000,000 cells per ml of the bioreactor vessel. In some embodiments, the bioreactor is inoculated with 50,000 to 5,000,000 cells per ml of the bioreactor vessel; 150,000 to 1,000,000 cells per ml of the bioreactor vessel; 200,000 to 500,000 cells per ml of the bioreactor vessel; or 250,000 to 500,000 cells per ml of the bioreactor vessel. In some embodiments, the bioreactor is inoculated with 200,000 to 400,000 cells per ml of the bioreactor vessel.

The stirred tank bioreactor comprises a cell culture medium. The cell culture medium facilitates cell expansion by providing necessary nutrients to the cells. Any suitable cell culture medium known in the art may be used, according to the cell type that is being expanded.

In one embodiment, the bioreactor is a batch bioreactor. In one embodiment, the bioreactor is a fed-batch bioreactors. In one embodiment, the bioreactor is a continuous (perfusion) bioreactor.

In one embodiment, the cell culture medium is a serum-free mammalian culture medium. In one embodiment, the cell culture medium is any suitable stem cell maintenance medium, such as mTeSR™ or E8 medium. Culture medium supplementation can include mTeSR plus and B8 media with Nutrient Mixture F12 (DMEM F12) as basal medium. An exemplary culture medium is described in Kuo et al. 2020, which is herein incorporated by reference (Kuo, H. H., Gao, X., DeKeyser, J. M., Fetterman, K. A., Pinheiro, E. A., Weddle, C. J. & Burridge, P. W. (2020). Negligible-cost and weekend-free chemically defined human iPSC culture. Stem Cell Reports, 14(2), 256-270).

The conditions within the cell culture vessel may also be controlled to facilitate cell expansion, for example pH, nutrient supply, toxic by-product disposal and dissolved oxygen (DO) levels may be controlled. Any suitable conditions known in the art may be used, according to the cell type that is being expanded.

In one embodiment, the nutrient supply comprises glucose, optionally within the range of 5 to 30 nM. In one embodiment, the nutrient supply comprises glutamine (or similar compounds such as GLUTAMAX™), optionally within the range of 1 to 10 nM.

In one embodiment, the toxic by-products comprise lactate, optionally wherein the level of lactate in the cell culture vessel is controlled such that the concentration of lactate does not exceed 60 mM. In one embodiment, the toxic by-products comprise ammonia, optionally wherein the level of ammonia in the cell culture vessel is controlled such that the concentration of ammonia does not exceed 5 mM.

In one embodiment, the pH of the cell culture is above pH 6. In another embodiment the pH of the cell culture is pH 6 to 7.5. In a preferred embodiment, the pH of the cell culture is 7 to 7.5, more preferably 7.2. In another preferred embodiment, the pH of the cell culture is 7.35. In one embodiment, the nutrient supply comprises glucose and/or amino acids. In one embodiment, the toxic by-products comprise lactate and/or ammonia. In one embodiment, the DO is 5-80%, including 5-60%.

In one embodiment, the dissolved oxygen (DO) is in the range of 30 to 70%. In some embodiments, the dissolved oxygen (DO) is in the range of 40 to 60%. In a preferred embodiment, the dissolved oxygen (DO) is 50%.

In some embodiments, the cells are cultivated with pH controlled at 7 to 7.5 and dissolved oxygen (DO) controlled at 40 to 60%, optionally wherein the temperature is °C to 40 °C, preferably 37 °C.

In some embodiments, the cells are cultivated with pH controlled at 7.2 and dissolved oxygen (DO) controlled at 50%, optionally wherein the temperature is 35 °C to 40 °C, preferably 37 °C.

In some embodiments, the cells are cultivated with pH controlled at 7.35 and dissolved oxygen (DO) controlled at 50%, optionally wherein the temperature is 35 °C to 40 °C, preferably 37 °C.

Air and CO₂ are supplemented to the headspace. In one embodiment, headspace gassing is carried out at 15 to 25 mL/min of a mixture of air and 4% to 8% CO₂ including 5% CO₂. In embodiments, headspace gassing is carried out at 19 mL/min of a mixture of air and 5% CO₂.

In some embodiments, cell expansion is carried out in a stirred tank bioreactor, wherein the bioreactor is inoculated with 200,000 to 400,000 cells per ml of the bioreactor vessel, optionally wherein the bioreactor uses a single ‘elephant ear’ impeller.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring speed of 250 RPM and at the second stirring speed is 400 RPM, optionally wherein the bioreactor uses a single ‘elephant ear’ impeller.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring speed of 250 RPM, which is increased to a second stirring speed of 400 RPM, optionally at a rate of 6.25 to 20 RPM per hour and optionally wherein the bioreactor uses a single ‘elephant ear’ impeller.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring speed of 200 to 300 RPM which is increased during the expansion process to a second stirring speed of 300 to 500 RPM, wherein the first and second stirring speeds are different.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring speed of 200 to 300 RPM, which is increased to a second stirring speed of 300 to 500 RPM, optionally at a rate of 6.25 to 20 RPM per hour.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring power of between 3 W/m³ and 75 W/m³ and the second stirring power of 75 W/m³ and 255 W/m³, wherein the first and second stirring powers are different by at least 45 or 50 W/m³ and optionally wherein the bioreactor uses a single ‘elephant ear’ impeller.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring power of between 3.93 W/m³ and 74.52 W/m³ and the second stirring power of 74.52 W/m³ and 251.51 W/m³, wherein the first and second stirring powers are different and optionally wherein the bioreactor uses a single ‘elephant ear’ impeller.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring power of about 18 W/m³ including 18.19 W/m³ and the second stirring power of about 75 W/m³ including 74.52 W/m³, optionally wherein the bioreactor uses a single ‘elephant ear’ impeller.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring power of about 18 W/m³ including 18.19 W/m³, which is increased to a second stirring power of about 75 W/m³ including 74.52 W/m³, optionally at a rate of 2.35 W/m³ per hour and further optionally wherein the bioreactor uses a single ‘elephant ear’ impeller.

In some embodiments, cell expansion is carried out in a stirred tank bioreactor, wherein the bioreactor is inoculated with 200,000 to 400,000 cells per ml of the bioreactor vessel, and wherein the cells are stirred at a first stirring power of 9 to 32 W/m³ including 9.32 to 31.44 W/m³ which is increased during the expansion process to a second stirring power of 32 to 150 W/m³ including 31.44 to 145.55 W/m³, wherein the first and second stirring powers are different, optionally by at least 50 W/m³. In some embodiments, cell expansion is carried out in a stirred tank bioreactor, wherein the bioreactor is inoculated with 200,000 to 400,000 cells per ml of the bioreactor vessel, and wherein the cells are stirred at a first stirring power of 9 to 32 W/m³ including 9.32 to 31.44 W/m³, which is increased at a rate of 2.35 to 23.53 W/m³ per hour to a second stirring power of 32 to 150 W/m³ including 31.44 to 145.55 W/m³.

In some embodiments, cell expansion is carried out in a stirred tank bioreactor using a single ‘elephant ear’ impeller, wherein the working volume is 200 to 250 mL and the fluid density is 800 to 1,200 kg/m³ of aqueous solution, and wherein the bioreactor is inoculated with 200,000 to 400,000 cells per ml of the bioreactor vessel, optionally wherein the headspace gassing is carried out at 15 to 25 mL/min of a mixture of air and 5% CO₂.

In some embodiments, cell expansion is carried out in a stirred tank bioreactor using a single ‘elephant ear’ impeller, wherein the working volume is 200 to 250 mL and the fluid density is 1,000 kg/m³ of aqueous solution, and wherein the bioreactor is inoculated with 200,000 to 400,000 cells per ml of the bioreactor vessel, optionally wherein the headspace gassing is carried out at 19 mL/min of a mixture of air and 5% CO₂.

### Cell Properties

The process of the present invention produces cell aggregates, e.g. PSC aggregates, with surprising properties. In particular, the cell aggregates produced by the process of the invention have a reduced aggregate size compared to cell aggregates produced by an expansion process that does not comprise a dynamic stirring process, i.e., a process that does not comprise stirring, or comprises a static stirring process where the stirring speed does not change.

In one embodiment, the cell aggregates produced by the process of the invention are 190 to 210 μm in size. In one embodiment the cell aggregates are at least 5 μm smaller compared to cell aggregates produced by an expansion process that does not comprise a dynamic stirring process. In one embodiment the cell aggregates are at least 10%, such as 10%, 11%, 12%, 13%, 14% or 15% smaller in size compared to cell aggregates produced by an expansion process that does not comprise a dynamic stirring process.

In one embodiment, the cell aggregates are approximately the same size in that they have a size distribution of less than 15 μm. In one embodiment, the cell aggregates are homogenous in size.

In another embodiment, the cell aggregates produced by the process of the invention have increased circularity compared to cell aggregates produced by an expansion process that does not comprise a dynamic stirring process. In one embodiment, the cell aggregates have a circularity of 0.85 to 0.90. In one embodiment the cell aggregates are at least 0.015 more circular compared to cell aggregates produced by an expansion process that does not comprise a dynamic stirring process. In one embodiment the cell aggregates have at least a 4%, such as 4%, 5%, 6%, 7%, 8%, 9% or 10% increase in circularity compared to cell aggregates produced by an expansion process that does not comprise a dynamic stirring process.

In a further embodiment, the cells produced by the process of the invention have increased viability compared to cells produced by an expansion process that does not comprise a dynamic stirring process. In some embodiments, at least 90% of cells are viable after completion of the expansion process. Preferably, at least 95% of cells are viable after completion of the expansion process. In some embodiments, at least 90% of cells are viable after completion of the expansion process, wherein the duration of the dynamic stirring is 5 days in total. In some embodiments, 95% of cells are viable after day 2 of the expansion process, wherein the duration of the dynamic stirring is 5 days in total. In some embodiments, 95% of cells are viable after day 2 of the expansion process, and the viability of the cells does not alter more than 5% during the expansion process. In one embodiment, the process produces at least 10% more viable cells compared to cell aggregates produced by an expansion process that does not comprise a dynamic stirring process.

The process of the present invention produces cells, e.g. PSCs, with additional surprising properties when the process comprises the expansion of pluripotent cells.

In particular, the cells produced by the process of the invention have improved pluripotency compared to cells produced by an expansion process that does not comprise a dynamic stirring process, i.e., a process that does not comprise stirring, or comprises a static stirring process where the stirring speed does not change.

Pluripotency may be measured according to specific pluripotency markers such as TRA-1-81, TRA-1-60 and/or any other known markers in the art. Pluripotency markers include TRA-1-81, TRA-1-60, and SSEA-4. Additional germ-layer differentiation assays were performed, including analysis of various markers, such as Sox2, Pax6, Sox17, and CXCR4. High levels of expression of these markers indicates a high level of pluripotency and ability to differentiate into different lineages. A suitable assay for testing pluripotency markers is germ layer analysis performed by differentiating cell aggregates into different lineages using STEMdiff™ Trilineage Differentiation Assay Kit (StemCell Technologies).

In the present invention, in one embodiment, the cells produced by the process of the invention have improved pluripotency compared to cells produced by an expansion process that does not comprise a dynamic stirring process. In one embodiment, the cells have increased levels of one or more pluripotency markers compared to cells produced by an expansion process that does not comprise a dynamic stirring process. In one embodiment, said pluripotency markers are selected from TRA-1-81 and TRA-1-60. In one embodiment, said pluripotency markers are selected from TRA-1-81, TRA-1-60 and SSEA-4.

In one embodiment, at least 85% of the cells produced by an expansion process of the invention are TRA-1-81+, such as at least 85%, 86%, 87%, 88% or more. In one embodiment, at least 86% of the cells produced by an expansion process of the invention are TRA-1-60+, such as at least 86%, 87%, 88% or more. In one embodiment, at least 85% of the cells produced by an expansion process of the invention are TRA-1-81+, such as at least 85%, 86%, 87%, 88% or more, and at least 86% of the cells produced by an expansion process of the invention are TRA-1-60+, such as at least 86%, 87%, 88% or more.

In one embodiment, at least 90% of the cells produced by an expansion process of the invention are SSEA-4+, such as at least 90%, 91%, 92%, 93% or more. In one embodiment, at least 85% of the cells produced by an expansion process of the invention are TRA-1-81+, such as at least 85%, 86%, 87%, 88% or more, and at least 90% of the cells produced by an expansion process of the invention are SSEA-4+, such as at least 90%, 91%, 92%, 93% or more. In one embodiment, at least 90% of the cells produced by an expansion process of the invention are SSEA-4+, such as at least 90%, 91%, 92%, 93% or more, and at least 86% of the cells produced by an expansion process of the invention are TRA-1-60+, such as at least 86%, 87%, 88% or more. In one embodiment, at least 85% of the cells produced by an expansion process of the invention are TRA-1-81+, such as at least 85%, 86%, 87%, 88% or more, at least 90% of the cells produced by an expansion process of the invention are SSEA-4+, such as at least 90%, 91%, 92%, 93% or more, and at least 86% of the cells produced by an expansion process of the invention are TRA-1-60+, such as at least 86%, 87%, 88% or more.

In one embodiment, at least 70% of the differentiated cells obtained by an expansion process of the invention expressed Sox2, such as at least 70%, 71%, 72%, 73% or more.

In one embodiment, at least 45% of the differentiated cells obtained by an expansion process of the invention expressedPax6, such as at least 48%, 49%, 50%, 51%, 52%, 53% or more.

In one embodiment, at least 80% of the differentiated cells obtained by an expansion process of the invention expressed Sox17, such as at least 81%, 82%, 83%, 84%, 85%, 86% or more.

In one embodiment, at least 80% of the differentiated cells obtained by an expansion process of the invention expressed CXCR4, such as at least 81%, 82%, 83%, 84%, 85%, 86% or more.

In one embodiment, at least 85% of the differentiated cells obtained by an expansion process of the invention expressTRA-1-81, such as at least 85%, 86%, 87%, 88% or more, at least 90% of the differentiated cells obtained by an expansion process of the invention express SSEA-4, such as at least 90%, 91%, 92%, 93% or more, at least 86% of the differentiated cells obtained by an expansion process of the invention express TRA-1-60, such as at least 86%, 87%, 88% or more, at least 70% of the differentiated cells obtained by an expansion process of the invention express Sox2, such as at least 70%, 71%, 72%, 73% or more, at least 45% of the differentiated cells obtained by an expansion process of the invention express Pax6, such as at least 48%, 49%, 50%, 51%, 52%, 53% or more, at least 80% of the differentiated cells obtained by an expansion process of the invention express Sox17, such as at least 81%, 82%, 83%, 84%, 85%, 86% or more, and at least 80% of the differentiated cells obtained by an expansion process of the invention expressCXCR4, such as at least 81%, 82%, 83%, 84%, 85%, 86% or more. Pluripotency may also be measured according to specific gene markers (transcriptome profile) such as KLF4, MYC, NANOG, SOX2 and POU5F1 and/or any other known gene markers in the art. High levels of these markers indicates a high level of pluripotency.

A suitable assay is gene expression profiling using RNA-Seq (RNA sequencing) as described in Example 3.

In one embodiment, pluripotency gene profile expression markers are selected from KLF4, MYC, NANOG, SOX2 and POU5F1. Exemplary relative expression levels are provided in FIG. 5.

It will be understood that references to RPM in relation to the stirring speed as disclosed herein covers equivalent RPM values depending on the bioreactor in use, to achieve the same stirring speed.

In some embodiments, the stirring speeds are 0.48 to 72 W/m³. In some embodiments, the stirring speed is altered in the range of 0.48 to 72 W/m³. In some embodiments, each stirring speed is selected from the range of 0.48 to 72 W/m³.

In some embodiments, the first stirring speed is 0.48 to 3.8 W/m³. In a preferred embodiment, the first stirring speed is 2.20 W/m³.

In some embodiments, the second stirring speed is 3.8 to 72 W/m³. In a preferred embodiment, the second stirring speed is 9 W/m³.

In some embodiments, the first stirring speed is 0.48 to 3.8 W/m³ which is increased during the expansion process to a second stirring speed of 3.8 to 72 W/m³, wherein the first and second stirring speeds are different.

In some embodiments, the first stirring speed is less than 3.8 W/m³ and the second stirring speed is more than 3.8 W/m³.

In some embodiments, the first stirring speed is 2.20 W/m³ and the second stirring speed is 9 W/m³.

In some embodiments, the first stirring speed is increased to the second stirring speed at a rate of 0.3 to 3 W/m³ per hour. In a preferred embodiment, the first stirring speed is increased to the second stirring speed at a rate of 0.3 W/m³ per hour.

In some embodiments, the first stirring speed is 0.48 to 3.8 W/m³, which is increased at a rate of 0.3 to 3 W/m³ per hour to a second stirring speed of 3.8 to 72 W/m³.

In a preferred embodiment, the first stirring speed is 2.20 W/m³, which is increased at a rate of 0.3 W/m³ per hour to a second stirring speed of 9 W/m³.

In one embodiment, the process comprises:
i) inoculating the bioreactor with a single-cell sample,
ii) dynamically stirring the cells in the bioreactor, wherein:
   a) the cells are stirred at a first stirring speed, optionally of 2.20 W/m³ from day 0 to day 2,
   b) the first stirring speed is increased to a second stirring speed, optionally at a rate of 0.3 W/m³ per hour from day 2 to day 3, and
   c) the cells are stirred at a second stirring speed, optionally of 9 W/m³ from day 3 to day 5,
iii) isolating the expanded cell culture after 5 days.

### Uses of the Present Invention

The present invention provides a novel process for the large-scale manufacture of cells, e.g. PSCs, with improved quality and viability. The cell products of the process of the invention may be used in a variety of industries.

In one embodiment, the expanded cell culture is suitable for use as a food product. In one embodiment, the expanded cell culture is suitable for use in the manufacture of a food product.

In one embodiment, the expanded cell culture is suitable for use as a medicine. In one embodiment, the expanded cell culture is suitable for use in the development of a medicine.

The invention also provides an expanded cell culture, e.g. PSC cell culture, produced according to the process as described anywhere herein.

### FIGURES

FIG. 1: Schematic of dynamic stirring speed protocol for 3D PSC aggregate culture. Stirred tank bioreactor was inoculated with single PSCs originating from 2D cultures in mTeSR™ plus medium. The bioreactor culture was initiated with a stirring speed of 250 RPM. The static culture remained using 250 RPM culture until the end of the cultivation. Meanwhile, the stirring speed in the dynamic culture was ramped up from Day 2 up to 400 RPM on Day 3.

FIG. 2: Dynamic stirring speed improves the 3D culture condition of hPSC. A. PSC aggregate size in 3D culture maintained at a constant speed (Static) or following a gradient speed increase (Dynamic). B. PSC aggregate circularity in 3D culture. Aggregate circularity was assessed by microscopic analysis, where aggregates with a circularity score between 0.8 and 1 were targeted, with 1 being a perfect sphere. C. PSC aggregate viability. D. Ratio of viable PSC cells positive for the pluripotency marker TRA-1-60. E. Ratio of viable PSC cells positive for the pluripotency marker TRA-1-81. F. Percentage of cell population positive for SSEA-4 marker in Static or Dynamic bioreactor expansion. Data are shown as mean±SEM of triplicate measurements and are representative of two independent experiments. Two-tailed unpaired t-tests is used for statistical analysis, where *p<0.05.

FIG. 3: Microscopic images of cell aggregates in different expansion conditions (Static/Dynamic) and different culture days (D1: Day 1; D7: Day 7).

FIG. 4: Markers obtained in germ layer analysis from Static or Dynamic bioreactor expansion.

FIG. 5: Gene expression profile (3D Dynamic vs 2D monolayer ‘control’) obtained by RNA-Sequencing. For each gene, bar on left=2D ‘control’, bar on right=3D dynamic.

FIG. 6: Conversion of stirring speed (RPM) to stirring power (W/m³) for AMBR250 single impeller “Elephant-Ear”.

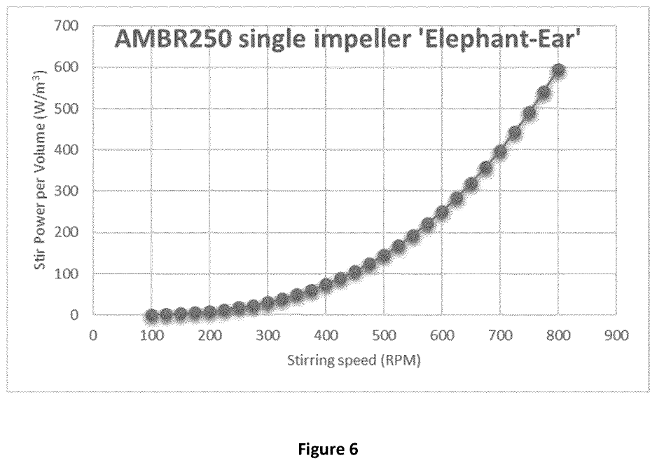

### EXPERIMENTAL SECTION

### Example 1

### Pluripotent Stem Cells Expansion in Stirred-Tank Bioreactor

### Methods

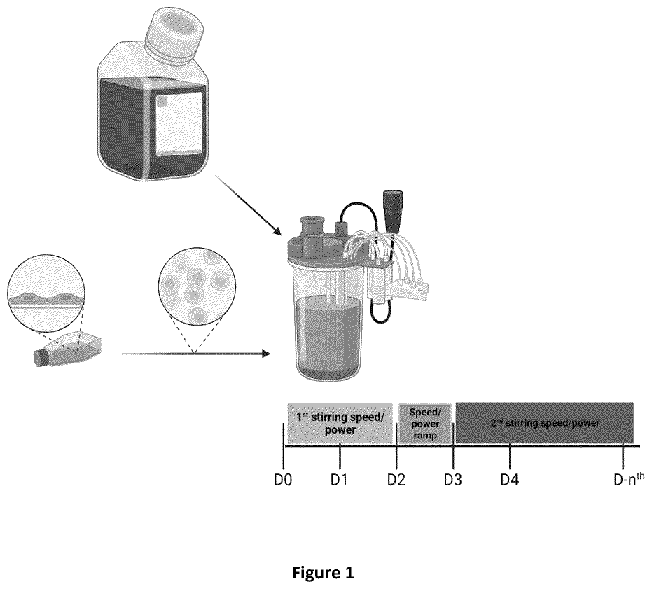

Cells were harvested from 2D T-flasks to seed the bioreactor. Prior, 2D cultured-cells in the T-flasks were dissociated with Accutase (ThermoFisher; 00-4555-56). The stirred-tank bioreactor (Ambr® 250 modular and mammalian culture vessel with elephant-ear impeller, Sartorius Stedim) was used to inoculate the single-cell suspension with the starting viable cell density of 200,000 or 400,000 cells per milliliter in mTeSR™ plus (StemCell Technologies; 100-0276)+10 μM Y-27632 ROCK Inhibitor (Abcam; 120129). The cells were cultivated at 37 °C with pH and dissolved oxygen (DO) controlled at 7.35 and 50%. The cultivation was initiated with a static stirring speed of 250 RPM for the first 48 hours. After 48 hours, the dynamic stirring speed was initiated according to Table 1. From 72 hours onwards, the stirring speed was maintained at 400 RPM until the end of the cultivation (FIG. 1). Media exchange was performed twice daily, approximately 8-12 hours apart from one another. About 80% of the media was refreshed in each media exchange.

TABLE 1
Dynamic stirring speed in the Ambr ® 250 stirred-tank bioreactor. The
stirring speed only applies for Ambr ® 250 system with elephant-ear
impeller (mammalian culture vessel). The stirring speed can be converted
into a universal variable for any type of stirred-tank bioreactor
(Power per volume; P/V). In the conversion, Newton number (Ne)
specific for elephant-ear vessel of 0.64 was used. The P/V was
calculated based on the formula reported by Rotondi, M et al. 2021
(incorporated herein by reference). Table 2 is a conversion table. FIG.
6 is a graph showing conversion from RPM to W/m³.

| | | | | | | |
|---|---|---|---|---|---|---|
| Day | 0 | 2 | 2 | 2 | 2 | 3 |
| Time (hr) | 0 | 48 | 54 | 60 | 66 | 72 |
| Ns (RPM) | 250 | 250 | 310 | 340 | 370 | 400 |
| P/V | 18.19 | 18.19 | 34.69 | 45.76 | 58.98 | 74.52 |
| (W/m³) | | | | | | |

TABLE 2

| Stirring speed | PPV |
|---|---|
| (RPM) | (W/m³) |
| 100 | 1.16 |
| 125 | 2.27 |
| 150 | 3.93 |
| 175 | 6.24 |
| 200 | 9.32 |
| 225 | 13.26 |
| 250 | 18.19 |
| 275 | 24.22 |
| 300 | 31.44 |
| 325 | 39.97 |
| 350 | 49.92 |
| 375 | 61.4 |
| 400 | 74.52 |
| 425 | 89.38 |
| 450 | 106.1 |
| 475 | 124.79 |
| 500 | 145.55 |
| 525 | 168.49 |
| 550 | 193.72 |
| 575 | 221.36 |
| 600 | 251.51 |
| 625 | 284.27 |
| 650 | 319.77 |
| 675 | 358.1 |
| 700 | 399.38 |
| 725 | 443.72 |
| 750 | 491.22 |
| 775 | 542 |
| 800 | 596.16 |

### Results

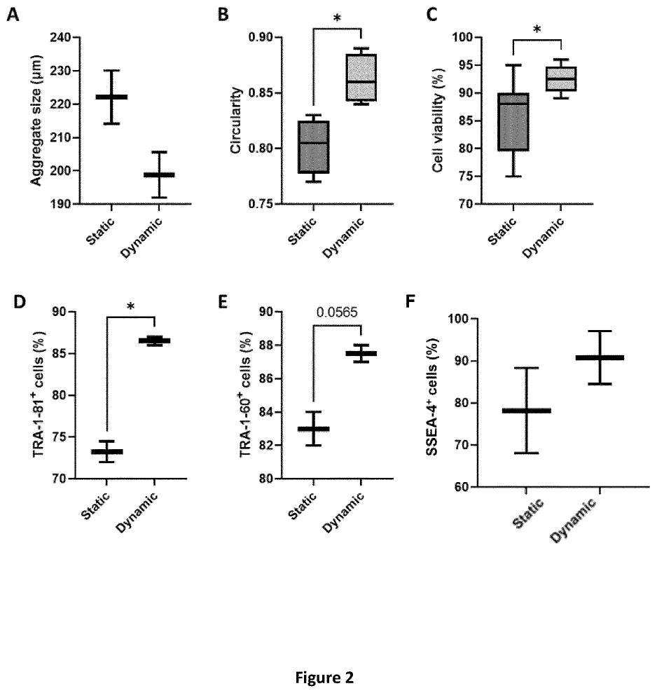

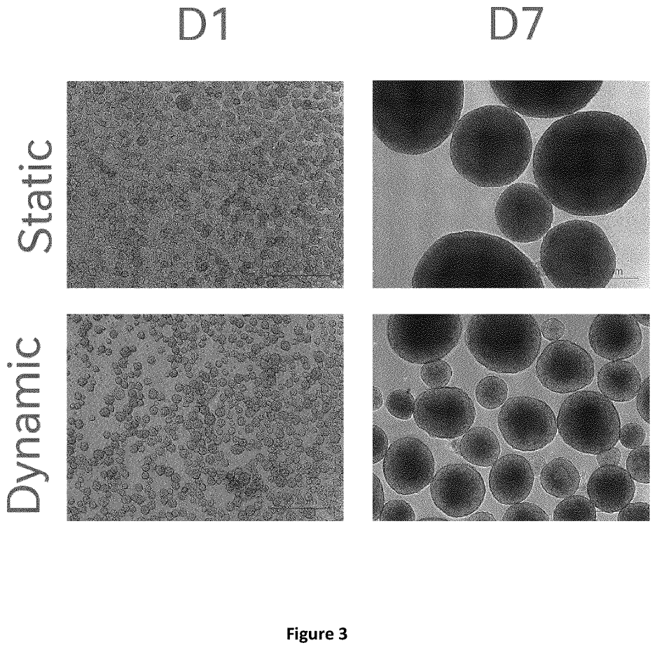

The present invention describes a novel strategy to improve the pluripotency of 3D cultured hiPSC aggregates in a stirred-tank bioreactor (FIG. 1). The strategy includes a range of bioreactor seed densities, an initial stirring speed (i.e., stirring power input/volume), and a gradient increase in the stirring speed from day 2 onwards. The presented method lowers the aggregate size (FIG. 2A), maintains the aggregate structure more homogeneous using the aggregate circularity index (FIG. 2B), and improves the cell viability (FIG. 2C). Cell aggregate in different expansion conditions (Static/Dynamic) and different culture days (Day 1; Day 7) are shown in FIG. 3. Consequently, the enhanced physical properties of the aggregates lead to improved cellular pluripotency by higher expression of the pluripotency markers TRA1-60 and TRA1-81 (FIG. 2 D-E), as well as pluripotency marker SSEA-4 (FIG. 2F).

### Example 2

3D Bioreactor Culture of iPSC and Three Germ Layer Analysis

### Methods

Human induced pluripotent stem cells (h-iPSCs) were cultured in a 250 mL AMBR® HTP bioreactor system, specifically the single impeller ‘elephant-ear’ vessel type, using B8 media. Bioreactors were seeded with single cell iPSC in suspension obtained from the 2D monolayer culture (e.g. T-flask), with the starting stirring speed of 175 RPM. iPSC cell aggregate was formed roughly 12-24 hours after the start of the bioreactor culture. Dynamic stirring condition was initiated 48 hours after the start of the culture, reaching a final stirring speed of 400 RPM in 24 hours. Aggregates of cells were cultured in the bioreactor for 5-7 days. Afterwards, intact cell aggregates were transferred into 5 mL working volume of well plates for germ layer analysis. Germ layer analysis was performed by differentiating the cell aggregates into different lineages. This analysis was performed by using STEMdiff™ Trilineage Differentiation Assay Kit (StemCell Technologies).

### Results

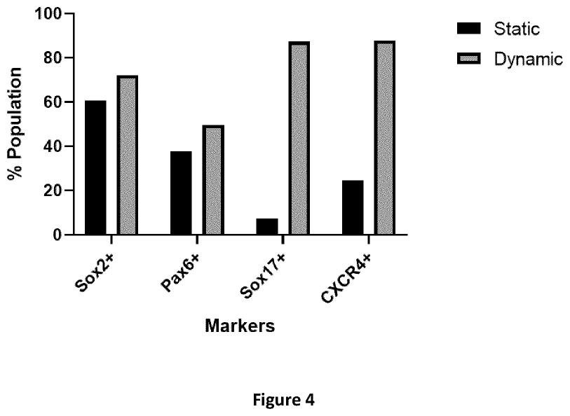

Intact aggregates, obtained from static/dynamic expansion protocol in bioreactor, were transferred to well plates for germ layer assay. These aggregates were exposed to lineage specific differentiation media. Various markers were observed in the germ layer analysis including Sox2, Pax6, Sox17 and CXCR4 (FIG. 4). The results indicated that the intact aggregates, expanded with dynamic protocol, can be differentiated more efficiently into various lineages.

### Example 3

### Gene Expression Profiling Using RNA-Seq Methods

Total RNA was extracted using the Agencourt RNAdvance Tissue Kit (Beckman Coulter). Tissues were disrupted and lyzed using the Fastprep 2×1′ at speed 6. 400 μL of lyzate was extracted and eluted in 50 μL. The RNA was quantified using Quant It Ribogreen assay (Life Technologies) and its quality was checked on a Fragment Analyzer. The Stranded mRNA Library Prep protocol convert the mRNA in a total RNA sample into a library of template molecules of known strand origin using the reagents provided in an Illumina® Stranded mRNA library prep workflow. 300 ng of total RNA was used of each RNA sample. A bead-based mRNA capture followed by a fragmentation, first and second strand synthesis, an end repair and a 3′ adenylation and indexed linker ligation was done. The adapter ligated cDNA library fragments were then enriched by PCR using 13 cycles of amplification. Indexed libraries were sequenced paired end (PE) with 59 bases each read on 3 and 1 individual P3 100 NextSeq2000 flow cells.

Raw counts were obtained from sequences by mapping to the human reference genome using STAR v.2.5.3 (Dobin A, 2012) and counting using htseq-count v.0.6.1 (Anders S, 2014).

Features were filtered to remove genes that are lowly expressed. Genes with very low counts are unlikely to be differentially expressed between groups. Lowly expressed genes were filtered by selecting only genes with at least 30 reads in at least 3 samples.

The filtering step is done on the CPM values which takes the library size into account. Here, it corresponds to a threshold of 1.012 on the CPM values.

Genes with no annotation were also discarded. With these filtering criteria, 13600 features were kept. Trimmed Mean of M-value (TMM) normalization (Robinson, M. D. & Oshlack, A 2010, A scaling normalization method for differential expression analysis of RNA-seq data. Genome biology, 11(3), 1-9), was used to account for composition bias between libraries. Differential expression analysis between different groups was performed using edgeR v3.40.2 (Robinson & Oshlack, 2010), which fits a negative binomial log-linear model to the normalized counts for each feature. Empirical Bayes methods are used to moderate the degree of overdispersion. The negative binomial model was further extended with quasi-likelihood method to account for gene-specific variability. Differential expression was tested by quasi-likelihood F-tests and resulting p-values were adjusted for multiple testing using Benjamini and Hochberg method (BH).

### Results

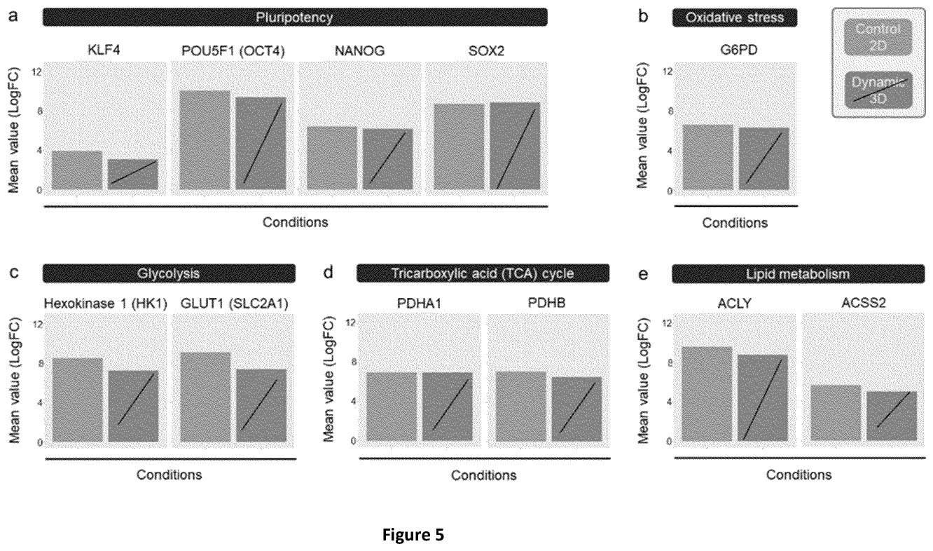

The gene expression profile of the cells cultured under 3D dynamic conditions was not significantly different from the gene expression profile of the cells cultured under 2D monolayer conditions (control). FIG. 5 shows the similar gene expression profile between 3D dynamic and 2D monolayer cell culture conditions for pluripotency genes (KLF4, POU5F1, NANOG, and SOX2), glucose metabolism genes (HK1, GLUT1), G6PD, TCA Cyce genes (PDHA1 and PDHB), and lipid metabolism genes (ACLY, ACSS2).

1: A process for expanding mammalian cells in a stirred tank bioreactor, the bioreactor comprising the mammalian cells and a cell culture medium, the process comprising dynamically stirring the mammalian cells in the tank bioreactor to produce an expanded cell culture.

2: The process of claim 1, wherein the dynamic stirring comprises stirring the mammalian cells at a stirring speed that is altered during production of the expanded cell culture.

3: The process of claim 2, wherein the stirring speed is increased from a first stirring speed to a second stirring speed.

4: The process of claim 2, wherein the first and second stirring speeds are in the range of 150 to 800 RPM.

5: The process of claim 3, wherein the first stirring speed is increased to the second stirring speed at a rate of 6.25 to 20 RPM per hour.

6: The process of claim 4, wherein the first stirring speed is 150 RPM to 300 RPM, and the second stirring speed is 400 RPM to 500 RPM.

7: The process of claim 5, wherein the first stirring speed is 250 RPM.

8: The process of claim 2, wherein the first and second stirring powers are in the range of 3 to 600 W/m³.

9: The process of claim 8, wherein the first stirring power is increased to the second stirring power at a rate of 2.35 to 23.53 W/m³ per hour.

10: The process of claim 9, wherein the first stirring power is 3 W/m³ to 32 W/m³, and the second stirring power is 70 W/m³ to 150 W/m³.

11: The process of claim 10, wherein the first stirring power is about 18 W/m³.

12: The process according to claim 1, wherein the dynamic stirring of the process has a duration of 3 to 30 days.

13: The process according to claim 2, wherein the stirring speed is altered from day 2 to day 3, where the process is initiated at day 0.

14: The process according to claim 1, wherein the mammalian cells for expansion are human cells, and/or the mammalian cells for expansion are stem cells.

15: The process according to claim 1, wherein the mammalian cells for expansion are selected from the group consisting of pluripotent stem cells (PSCs), muscle stem cells/satellite cells (MuSC/SC), adipose derived stem cells (ADSC), adipocytes, epithelial cells, mesenchymal stem/stromal cells (MSCs), fibro-adipogenic progenitors (FAPS), induced-pluripotent stem cells (iPSCs), breast milk stem cells (BMSCs), embryonic-like stem cells (ELC-Cs), chemically induced pluripotent stem cells (CiPSCs), chemically induced totipotent stem cells (CiTotiSCs), and mixtures thereof.

16: The process according to claim 1, wherein the process comprises:
i) inoculating the tank bioreactor with a single-cell sample,
ii) dynamically stirring the mammalian cells in the tank bioreactor, and
iii) isolating the expanded cell culture.

17: The process according to claim 1, wherein the process comprises:
i) inoculating the tank bioreactor with a single-cell sample,
ii) dynamically stirring the mammalian cells in the tank bioreactor, wherein:
a) the mammalian cells are stirred at a first stirring speed,
b) the first stirring speed is increased to a second stirring speed, and
c) the mammalian cells are stirred at a second stirring speed, and
iii) isolating the expanded cell culture after 5 days.

18: The process according to claim 1, wherein the process comprises:
i) inoculating the tank bioreactor with a single-cell sample,
ii) dynamically stirring the mammalian cells in the tank bioreactor, wherein:
a) the mammalian cells are stirred at a first stirring power,
b) the first stirring power is increased to a second stirring power, and
c) the mammalian cells are stirred at a second stirring power, and
iii) isolating the expanded cell culture after 5 days.

19: The process according to claim 1, wherein:
the process produces cell aggregates, wherein the process decreases cell aggregate size and/or circularity of the cell aggregates compared to a process which does not comprise dynamic stirring, and/or
wherein the process increases cell viability compared to a process which does not comprise dynamic stirring, and/or
wherein the mammalian cells for expansion are pluripotent cells, and the process increases cell pluripotency compared to a process which does not comprise dynamic stirring.

20: The process according to claim 1, wherein the expanded cell culture is suitable for use as a food product or as a medicine.

21: An expanded cell culture produced according to claim 1.
